(19) United States
(12) Patent Application Publication
(10) Pub. No.: US 2023/0257434 A1
(43) Pub. Date: Aug. 17, 2023

(54) CELL-DERIVED VESICLES COMPRISING TARGET PROTEIN AND METHOD FOR PRODUCING SAME

(71) Applicants: MDimune Inc., Seoul (KR); Seoul National University R&DB Foundation, Seoul (KR)

(72) Inventors: Shin Gyu Bae, Gyeonggi-do (KR); Seung Wook Oh, Gyeonggi-do (KR); Joong Hoon Park, Gangwon-Do (KR); Tae Sub Park, Gangwon-Do (KR)

(21) Appl. No.: 17/758,825

(22) PCT Filed: Jan. 13, 2021
(86) PCT No.: PCT/KR2021/000475

§ 371 Date: Jul. 14, 2022

(30) Foreign Application Priority Data
Jan. 14, 2020 (KR) 10-2020-0005014
Nov. 10, 2020 (KR) 10-2020-0149241

(51) Int. Cl. C07K 14/47; C12N 5/07; C07K 14/705; A61K 38/17; A61P 21/00

(52) U.S. Cl. CPC C07K 14/4722; C12N 5/06; C07K 14/70596; A61K 38/17; A61P 21/00; C12N 2510/00

(57) ABSTRACT

The present invention relates to cell-derived vesicles comprising target protein Prokineticin receptor 1 (PROKR1), and a therapeutic agent for muscle diseases comprising the same. When applied to myoblasts, the cell-derived vesicles comprising PROKR1 according to the present invention promote muscle differentiation and induce differentiation into myotubes, and can thus be used to prevent or treat muscle diseases and can be widely used in the pharmaceutical industry and the field of health functional foods.

### CROSS-REFERENCE TO RELATED APPLICATIONS

This is a U.S. National Phase of International Application PCT/KR2021/000475, filed Jan. 13, 2021, which claims priority to Korean Patent Application No. 10-2020-0005014, filed Jan. 14, 2020 and Korean Patent Application No. 10-2020-0149241, filed Nov. 10, 2020.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “58164_SubSeqListing.txt”, which was created on Dec. 8, 2022 and is 11,269 bytes in size. The subject matter of the Sequence Listing is incorporated herein in its entirety by reference.

### TECHNICAL FIELD

The present invention relates to a cell-derived vesicle comprising a target protein, PROKR1 (Prokineticin receptor 1) and a therapeutic agent for a muscle disease comprising the same.

### BACKGROUND ART

Microvesicles are a type of organelle that generally has a size of 0.03 μm to 1 μm and are naturally separated from the cell membrane in almost all types of cells to have a double phospholipid membrane, which is the structure of the cell membrane. These vesicles are basic cell tools for metabolism, transport of metabolites, enzyme storage, chemical reactions, etc. They are known to play a mediating role in cell-to-cell signaling by delivering mRNA, miRNA, and protein between cells.

Meanwhile, prokineticin is a secretory, multifunctional chemokine-like peptide. Prokineticin performs biological functions by interacting with two G-protein coupled receptors (GPCRs) called prokineticin receptor 1 (PROKR1) and prokineticin receptor 2 (PROKR2). PROKR1 and PROKR2 share an overall amino acid sequence homology of about 87%. PK1 and PK2 interact with PROKR1 and PROKR2, respectively. At the cellular level, activation of prokineticin receptors leads to calcium mobilization, stimulation of phosphoinositide turnover, and activation of the MAP kinase signaling pathway. At the multicellular level, prokineticin exhibits angiogenic activity and induces cell proliferation and migration. Prokineticin and receptors thereof have been implicated in the development of several human cancers and have also been shown to be involved in the nociception and transmission of pain. The prokineticin signaling system suggests a potential target for treating and/or preventing a variety of diseases and disorders.

### DISCLOSURE

### Technical Problem

While searching for and researching substances effective for treating a muscle disease, the present inventors prepared cells overexpressing PROKR1 and confirmed that cell-derived vesicles prepared from cells overexpressing PROKR1 contain PROKR1 at a high level and it is effective in treating muscle diseases, thereby completing the present invention.

Accordingly, an object of the present invention is to provide a pharmaceutical composition for preventing or treating muscle disease comprising a cell-derived vesicle overexpressing PROKR1.

In addition, another object of the present invention is to provide a method for preventing or treating a muscle disease, comprising step of treating a cell-derived vesicle overexpressing PROKR1 to an individual in need thereof.

### Technical Solution

In order to achieve the above object, the present invention provides a cell-derived vesicle comprising PROKR1 (Prokineticin receptor 1) or a gene encoding the same.

In addition, the present invention provides a reagent composition for delivering PROKR1 protein or a gene encoding the same into cells, comprising a cell-derived vesicle containing PROKR1 (Prokineticin receptor 1) or a gene encoding the same.

In addition, the present invention provides a reagent composition for promoting myogenic differentiation, the composition comprising a cell-derived vesicle containing PROKR1 (Prokineticin receptor 1) or a gene encoding the same.

In addition, the present invention provides a pharmaceutical composition for preventing or treating a muscle disease, the composition comprising a cell-derived vesicle containing PROKR1 (Prokineticin receptor 1) or a gene encoding the same.

In addition, the present invention provides a health functional food composition for preventing or improving a muscle disease, the composition comprising a cell-derived vesicle containing PROKR1 (Prokineticin receptor 1) or a gene encoding the same.

In addition, the present invention comprises a method for preventing or treating a muscle disease, the method comprising step of treating a cell-derived vesicle overexpressing PROKR1 to an individual in need thereof.

### Advantageous Effects

The cell-derived vesicle comprising PROKR1 (Prokineticin receptor 1) according to the present invention is treated to myoblasts or myotube cells to promote myoblast differentiation and induces differentiation into myotube cells, thereby preventing or treating muscle diseases and thus may be widely used in the pharmaceutical industry and health functional food fields.

### DESCRIPTION OF DRAWINGS

FIG. 1 is a view showing cDNA sequence information of the human PROKR1 gene.

FIG. 2 is a schematic view showing a PROKR1 overexpression vector.

FIG. 3 is a view showing the results of PROKR1/PROKR1 gene editing.

FIG. 4 is a view showing gene expression in the PROKR1/PROKR1 gene-deficient cells (***p<0.001 vs. WT, Dunnett's multiple comparison test).

FIG. 5 is a schematic view showing the manufacturing process of a cell-derived vesicle using the micro-extrusion method and density gradient ultracentrifugation.

FIG. 6 is a view showing the results of analyzing the purity and size of cell-derived vesicles analyzed using size exclusion chromatography (blue arrow=OptiPrep gradient solution. abscissa=retention time (min), ordinate=absorbance (mAU)).

FIG. 7 is a view showing the results of particle size analysis of cell-derived vesicles.

FIG. 8 is a view showing the expression of a marker protein in cell-derived vesicles (GFP 293T cells=protein extract of GFP-overexpressing HEK293T cells, Naive 293T cells=protein extract of non-engineered HEK293T cells).

FIG. 9 is a view showing the total RNA profile of a cell-derived vesicle (black arrow=18S rRNA, blue arrow=28S rRNA).

FIG. 10 is a view showing the detection of a green fluorescence signal of GFP protein from a cell-derived vesicle smear sample in order to confirm GFP expression in cell-derived vesicles of GFP-overexpressing HEK293T cells.

FIG. 11 is a view confirming the expression of GFP mRNA in HEK293T cells, GFP-overexpressing HEK293T cells, cell-derived vesicles of HEK293T cells, and cell-derived vesicles of GFP-overexpressing HEK293T cells through RT-PCR.

FIG. 12 is a view showing green fluorescence expression observed up to 24 hours after treatment with GFP-overexpressing cell-derived vesicles in order to confirm intercellular transport of the cell-derived vesicle containing the GFP protein (arrow=an example of the GFP protein-containing cell-derived vesicles, scale bar=200 μm).

FIG. 13 is a view showing the presence or absence of GFP mRNA for each time period by treating HEK293T cells with cell-derived vesicles of GFP-overexpressing HEK293T cells.

FIG. 14A is a view showing the change in cell proliferation capacity for C2C12 cells according to the treatment concentration of PROKR1 overexpressing cell-derived vesicles in treatment group 1 (*p<0.05, **p<0.01, ***p<0.001 vs. 0 μg/mL, Dunnett multiple comparison test).

FIG. 14B is a view showing the change in cell proliferation capacity for C2C12 cells according to the treatment concentration of PROKR1 overexpressing cell-derived vesicles in treatment group 2 (*p<0.05, **p<0.01, ***p<0.001 vs. 0 μg/mL, Dunnett multiple comparison test).

FIG. 15 is a diagram showing the size distribution of PROKR1-overexpressing cell-derived vesicles or PROKR1-deficient cell-derived vesicles.

FIG. 16 is a view showing the results of analyzing the expression of a marker protein in a protein extracted from a PROKR1-overexpressing cell and a PROKR1-deficient cell and a cell-derived vesicle thereof.

FIG. 17 is a view confirming the PROKR1 protein transport ability of a PROKR1-overexpressing cell-derived vesicle.

FIG. 18 is a microscopic view showing the effect of PROKR1-overexpressing cell-derived vesicles on myoblast differentiation and apoptosis when myoblasts were treated with the vesicles.

FIG. 19A is a view confirming the effect of PROKR1-overexpressing cell-derived vesicles on apoptosis after myoblast differentiation by flow cytometry.

FIG. 19B is a view showing a graph prepared based on the results of flow cytometry (*p<0.05).

FIG. 20 is a view confirming the effect of PROKR1-overexpressing cell-derived vesicles on the differentiation of myoblasts into myotube cells through phalloidin staining.

FIG. 21 is a view showing the results of measuring the expression of a muscle development marker gene in order to confirm the effect of PROKR1-overexpressing cell-derived vesicles on the muscle development ability (^{a}p<0.05, ^{b}p<0.001 vs. WT C2C12 at differentiation day 0 (WT Day 0), ^{c}p<0.01, ^{d}p<0.001 vs. WT Day 6, ^{e}p<0.01, ^{f}p<0.001 vs. C2C12^{Prokr1-/-} at differentiation day 0 (KO Day 0), Dunnett multiple comparison test).

FIG. 22A is a view showing the effect of PROKR1-overexpressing cell-derived vesicles on the insulin-stimulated glucose uptake capacity of wild-type C2C12 cells in which the gene is not deleted. (^{g}p<0.001 vs. untreated WT C2C12, ^{h}p<0.05 vs. untreated C2C12^{Prokr1-/-}, ⁱp<0.05 vs. insulin (INS)-treated C2C12^{Prokr1-/-}, ^{j}p<0.05 vs. INS+PROKR1^{Tg} CDVs-treated C2C12P^{Prokr1-/-}, Dunnett multiple comparison test).

FIG. 22B is a view showing the effect of PROKR1-overexpressing cell-derived vesicles on the insulin-stimulated glucose uptake capacity of PROKR1-deficient C2C12 cells ^{g}p<0.001 vs. untreated WT C2C12, ^{h}p<0.05 vs. untreated C2C12^{Prokr1-/-}, ⁱp<0.05 vs. insulin (INS)-treated C2C12^{Prokr1-/-}, ^{j}p<0.05 vs. INS+PROKR1^{Tg} CDVs-treated C2C12^{Prokr1-/-}, Dunnett multiple comparison test).

### BEST MODE

The present invention provides a cell-derived vesicle comprising PROKR1 (Prokineticin receptor 1) or a gene encoding the same.

As used herein, the term “cell-derived vesicle” refers to a vesicle generated from a cell and is generally a type of cell organelle, which is released from the cell membrane in almost all types of cells to form a double phospholipid membrane, which is the structure of the cell membrane.

The cell-derived vesicle of the present invention may have a micrometer size, for example, 0.03 μm to 1 μm. The vesicle of the present invention is separated from the inside and outside by a lipid bilayer composed of the cell membrane component of the derived cell and has cell membrane lipid, cell membrane protein, nucleic acid, and cell component and is smaller in size than the original cell, but not limited thereto.

The cell-derived vesicle of the present invention may be a cell-derived vesicle from which a vesicle may be prepared, and preferably, a cell-derived vesicle prepared from ‘PROKR1 protein (Prokineticin receptor 1) or a cell containing a gene encoding the same.’

In the present invention, the ‘PROKR1 (Prokineticin receptor 1) protein or a cell containing a gene encoding the same’ may be used without limitation as long as the cell that expresses the PROKR1 gene to produce the PROKR1 protein, and preferably it may be a cell transformed to express the PROKR1 protein by transducing the vector containing the PROKR1 gene into the cell.

In the present invention, the process of producing ‘a cell-derived vesicle comprising PROKR1 or a gene encoding the same’ comprises steps of preparing a transformed cell by introducing the gene encoding the PROKR1 protein; and preparing a cell-derived vesicle from the transformed cell.

In the present invention, for the ‘step of preparing a transformed cell by introducing the gene encoding the PROKR1 protein’, methods known in the art may be used without limitation as long as the vector containing the gene encoding the PROKR1 protein is transduced into the cell. Here, ‘transduction’ means that the PROKR1 gene is introduced into the host so that the PROKR1 gene becomes replicable as an extrachromosomal factor or by chromosomal integration completion. Transduction includes any method of introducing a nucleic acid molecule into an organism, cell, tissue or organ, and as known in the art, may be performed by selecting an appropriate standard technique according to the host cell. Such methods may include electroporation, calcium phosphate (CaPO₄) precipitation, calcium chloride (CaCl₂)) precipitation, microinjection, polyethylene glycol (PEG) method, DEAE (diethylaminoethyl)-dextran method, cationic liposome method, and lithium acetate-DMSO method and the like may be included, and may preferably be a transduction method using lipofectamine, but is not limited thereto.

In the present invention, the ‘step of preparing a cell-derived vesicle from the transformed cell’ is performed by using a method selected from the group consisting of extruding the suspension containing the transformed cell, sonication, cell lysis, homogenization, freeze-thaw, electroporation, chemical treatment, mechanical degradation and treatment of a physical stimulus applied externally to the cell, but not limited to.

Preferably, ‘the step of preparing a cell-derived vesicle from the transformed cell’ may comprise steps of: 1) micro-extruding the transformed cell; and 2) filtering.

In the ‘1) step of micro-extruding the transformed cells’, the extrusion force during micro-extrusion may be 5 to 200 psi, 10 to 150 psi, or 50 to 100 psi. In addition, the filter used in the micro-extrusion may be a filter of 0.01 μm to 20 μm, preferably 0.2 μm to 10 μm, and it may be a step of for micro-extrusion by sequentially using 1 to 10 filters that gradually decrease in size.

The ‘step of 2) filtering’ may be a step of filtering the micro-extruded extrudate through a filter of 0.1 μm to 0.51 μm.

In addition, the ‘step of preparing a cell-derived vesicle from the transformed cell’ may preferably further comprise step of ‘3) density gradient ultracentrifugation’, and step of ‘3) density gradient ultracentrifugation’ may be performed in 50,000G to 150,000G at 2° C. to 6° C. for 1 to 3 hours. If it is prepared by further comprising step of density gradient ultracentrifugation, cell-derived vesicles having a more uniform size may be formed, resulting in higher purity.

In the present invention, when ‘a cell-derived vesicle comprising PROKR1 (Prokineticin receptor 1) or a gene encoding the same’ is treated in a cell, PROKR1 or a gene encoding the same is transported to the cell, and it may be to express PROKR1 at a high level in the treated cell. Preferably, the PROKR1 protein may be maintained for 30 minutes to 24 hours after the cell-derived vesicle is treated in the cell, and the gene encoding the PROKR1 is maintained for 1 hour to 48 hours after the vesicle is treated in the cell, but is not limited thereto.

In the present invention, ‘the cell-derived vesicle comprising the PROKR1 (Prokineticin receptor 1) or a gene encoding the same’ may be a vesicle capable of having a muscle-generating ability and may be a vesicle capable of having a myogenic differentiation promoting ability. When it is treated to PROKR1-deficient cells, it may be possible to express late myogenic markers Mb, Myh7 or Myog and early myogenic markers Pax3, Pax7 or Myod at the level of cells in which PROKR1 is not deficient. Also, preferably, the myoblast differentiation promotion may be to promote differentiation from myoblasts to myotubes or myocytes.

In the present invention, the ‘cell-derived vesicle comprising PROKR1 (Prokineticin receptor 1) or a gene encoding the same’ is a vesicle capable of inhibiting apoptosis after myoblasts are differentiated into myotube cells or myocytes.

In the present invention, the ‘cell-derived vesicle comprising PROKR1 (Prokineticin receptor 1) or a gene encoding the same’ may be a vesicle capable of using to prevent, improve or treat muscle disease based on the muscle-generating ability, myogenic differentiation promoting ability and post-differentiation apoptosis inhibitory ability.

In the present invention, the ‘cell-derived vesicle comprising PROKR1 (Prokineticin receptor 1) or a gene encoding the same’ may be a vesicle capable of promoting glucose uptake of myotube cells by insulin stimulation and be a vesicle capable of improving insulin resistance by this mechanism and having a preventive or therapeutic effect on diabetes. It may preferably be a vesicle capable of promoting differentiation into myotube cells showing glucose uptake ability by insulin stimulation in cells with lost myogenic differentiation ability and may preferably be a vesicle capable of preventing or treating diabetes based on these effects

In the present invention, the ‘cell-derived vesicle comprising PROKR1 (Prokineticin receptor 1) or a gene encoding the same’ may be a vesicle capable of having an inner diameter of 161.7±12.3 nm (mean±standard deviation).

In the present invention, the ‘cell-derived vesicle comprising PROKR1 (Prokineticin receptor 1) or a gene encoding the same’ may be a vesicle capable of including a marker protein of a derived cell, and preferably the marker protein may be CD9 or CD81. In particular, the cell-derived vesicle may include marker proteins, preferably CD9 or CD81 in a higher ratio compared to the derived cell and may preferably include marker proteins three times or more compared to the derived cell.

In the present invention, the ‘cell-derived vesicle comprising PROKR1 (Prokineticin receptor 1) or a gene encoding the same’ may be one in which genes or RNAs of various sizes are conserved in complete form.

In the present invention, the ‘cell-derived vesicle comprising PROKR1 (Prokineticin receptor 1) or a gene encoding the same’ may be a vesicle capable of having a higher production rate of the cell-derived vesicle, the amount of protein and/or RNA in the cell-derived vesicle compared to ‘cell-derived vesicle manufactured using PROKR1 gene-deficient cells.’

In the present invention, the ‘cell-derived vesicle comprising PROKR1 (Prokineticin receptor 1) or a gene encoding the same’ may be a vesicle capable of being non-toxic to cells. It may preferably be non-toxic when treated at a concentration of 0.0001 to 100 μg/mL, more preferably be non-toxic when treated at a concentration of 0.0001 to 80 μg/mL, and even preferably be non-toxic when treated at a concentration of 0.0001 to 50 μg/mL.

In addition, the present invention provides a reagent composition for delivering PROKR1 protein or a gene encoding the same into cells, the composition comprising a cell-derived vesicle comprising PROKR1 (Prokineticin receptor 1) or a gene encoding the same.

In addition, the present invention provides a reagent composition for promoting myogenic differentiation, the composition comprising a cell-derived vesicle comprising PROKR1 (Prokineticin receptor 1) or a gene encoding the same.

In the present invention, the reagent composition may comprise the ‘cell-derived vesicle comprising PROKR1 (Prokineticin receptor 1) or a gene encoding the same’ at a concentration of 0.0001 to 100 μg/mL, preferably 0.0001 to 80 μg/mL, and more preferably 0.0001 to 50 μg/mL. When the reagent composition of the present invention comprises the cell-derived vesicle in excess concentration of 100 μg/mL, it may exhibit toxicity to cells.

In addition, the present invention provides a pharmaceutical composition for preventing or treating muscle disease, the composition comprising a cell-derived vesicle comprising PROKR1 (Prokineticin receptor 1) or a gene encoding the same.

The pharmaceutical composition of the present invention may promote glucose absorption of myotube cells by insulin stimulation, thereby improving insulin resistance and preventing or treating diabetes. Therefore, it may be one capable of simultaneously preventing or treating diabetes and muscle disease.

Therefore, the present invention provides a method for preventing or treating a muscle disease, the method comprising step of treating PROKR1-overexpressing cell-derived vesicles to an individual in need thereof.

In addition, the present invention provides the use of PROKR1-overexpressing cell-derived vesicles for preventing or treating a muscle disease and the use for the preparation of a pharmaceutical composition for preventing or treating a muscle disease. In the present invention, the muscle disease may be one selected from the group consisting of mitochondrial myopathy, encephalopathy, lactic acidosis, and stroke-like episodes (MELAS) syndrome, myoclonic epilepsy with ragged red fiber (MERRF) syndrome, Kearns-Sayre syndrome, myopathy, encephalomyopathy, myasthenia, myasthenia gravis, amyotrophic lateral sclerosis, muscular dystrophy, amyotrophia, muscular hypotonia, muscle weakness and muscular rigidity.

In the present invention, the cell-derived vesicle may be included in any amount (effective amount) depending on the formulation and purpose of mixing, as long as it may exhibit the prevention or treatment effect of muscle disease. Here, the term “effective amount” refers to the amount of active ingredient included in the composition of the present invention, which may have the intended functional and pharmacological effects, such as the effect of restoring myogenic differentiation, when the composition of the present invention is administered to a mammal, preferably a human subject to the application, during the administration period as suggested by a medical professional. Such an effective amount may be experimentally determined within the ordinary capacity of those skilled in the art, and it may be included preferably at a concentration of 1×10⁵ to 1×10¹³ particles/ml, more preferably 5×10⁷ to 1×10¹³ particles/ml, even more preferably 5×10⁸ to 1×10¹³ particles/ml, and still more preferably 1×10⁹ to 1×10¹³ particles/ml.

The pharmaceutical composition for preventing or treating muscle disease of the present invention, in addition to the active ingredient, may further include any compound or natural extract that has already been verified for safety and is known to have a preventive or therapeutic effect on muscle disease for the purpose of increasing/reinforcing the prevention or treatment effect of muscle disease.

The pharmaceutical composition of the present invention may be formulated and used in the form of oral dosage forms such as powders, granules, tablets, capsules, suspensions, emulsions, syrups, and aerosols, external preparations, suppositories, and sterile injection solutions, respectively, according to conventional methods. Carriers, excipients and diluents that may be included in the pharmaceutical composition include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate and mineral oil. The formulations are prepared using diluents or excipients (e.g., fillers, extenders, binders, humectants, disintegrants, surfactants, etc.) that are commonly used. Solid formulations for oral administration may include tablets, pills, powders, granules, capsules, etc. These solid formulations may be prepared by mixing the pharmaceutical composition of the present invention with at least one excipient (e.g., starch, calcium carbonate, sucrose, lactose, gelatin, etc.). In addition to simple excipients, lubricants (e.g., magnesium stearate and talc) may also be used. Liquid formulations for oral administration may contain suspensions, solutions for internal use, emulsions, syrups, etc. In addition to the simple diluents commonly used (e.g., water and liquid paraffin), various other excipients (e.g., humectants, sweeteners, fragrances, preservatives, etc.) may also be used. Formulations for parenteral administration contains sterile aqueous solutions, non-aqueous solvents, suspensions, emulsions, lyophilized preparations, and suppositories. As the non-aqueous solvents and the suspensions, propylene glycol, polyethylene glycol, vegetable oil (e.g., olive oil), an injectable ester (e.g., ethyloleate), etc. may be used. As a base for the suppositories, Witepsol, Macrogol, Tween 61, cacao butter, laurin butter, glycerogelatin, etc. may be used.

The dosage of the pharmaceutical composition of the present invention varies depending on the age, sex, and weight of the subject to be treated, the specific disease or pathological condition to be treated, the severity of the disease or pathological condition, the route of administration, and the judgment of the prescriber. Dosage determination based on these factors is within the level of one of ordinary skill in the art, and generally dosages range from 0.01 mg/kg/day to approximately 2000 mg/kg/day. A more preferred dosage is 1 mg/kg/day to 500 mg/kg/day. It may be administered once a day or may be administered in several divided doses. The above dosage does not limit the scope of the present invention in any way.

The pharmaceutical composition of the present invention may be administered to mammals such as mice, livestock, and humans with various routes. Any mode of administration may be envisaged, for example, by oral, rectal or intravenous, intramuscular, subcutaneous, intrauterine intrathecal or intracerebrovascular injection. Since the compound of the present invention has almost no toxicity and side effects, it is a drug that may be safely used even when taken for a long period of time for prophylactic purposes.

In addition, the present invention provides a health functional food composition for preventing or improving muscle disease, the composition comprising a cell-derived vesicle comprising PROKR1 (Prokineticin receptor 1) or a gene encoding the same.

The health functional food composition of the present invention may be one capable of promoting glucose absorption in myotubes by insulin stimulation, thereby improving insulin resistance and preventing or improving diabetes. Therefore, it may be one capable of simultaneously preventing or improving diabetes and muscle disease.

The health functional food of the present invention includes ingredients commonly added during food production, for example, proteins, carbohydrates, fats, nutrients and seasonings. For example, when manufactured as a drink, a flavoring agent or natural carbohydrate may be included as an additional ingredient in addition to the cell-derived vesicle as an active ingredient. For example, natural carbohydrates may include monosaccharides (e.g., glucose, fructose, etc.), disaccharides (e.g., maltose, sucrose, etc.), oligosaccharides, polysaccharides (e.g., dextrin, cyclodextrin, etc.), and sugar alcohols (e.g., xylitol, sorbitol, erythritol, etc.). As flavoring agents, natural flavoring agents (e.g., taumarin, stevia extract, etc.) and synthetic flavoring agents (e.g., saccharin, aspartame, etc.) may be included.

The health functional food of the present invention includes the form of tablets, capsules, pills, or liquids, and the food to which the cell-derived vesicle of the present invention can be added includes, for example, various drinks, meat, sausage, bread, candy, snacks, noodles, ice cream, dairy products, soups, ionic drinks, beverages, alcoholic beverages, gum, tea and vitamin complex.

Since the functional health food for preventing or improving muscle disease of the present invention has the same active ingredient and effect as the pharmaceutical composition for preventing or treating muscle disease, the description of common content between the two is excluded to avoid excessive complexity of the present specification.

### MODES OF THE INVENTION

The present invention will be described in detail through the following examples. These examples are only for illustrative purposes and do not limit the scope of the present invention.

### Example 1. Construction of Prokineticin Receptor 1 (PROKR1) Overexpression Vector

The cDNA (1,182 bp) of the human PROKR1 gene was synthesized based on the nucleotide sequence information (NM 138964) confirmed by the US National Center for Biotechnology Information (http://www.ncbi.nlm.nih.gov) (Bionics, Seoul, Korea). The synthesized PROKR1 gene was inserted into the piggyBac transposon transgene expression system vector (System Biosciences, Palo Alto, Calif.) to construct a PROKR1-overexpressing vector.

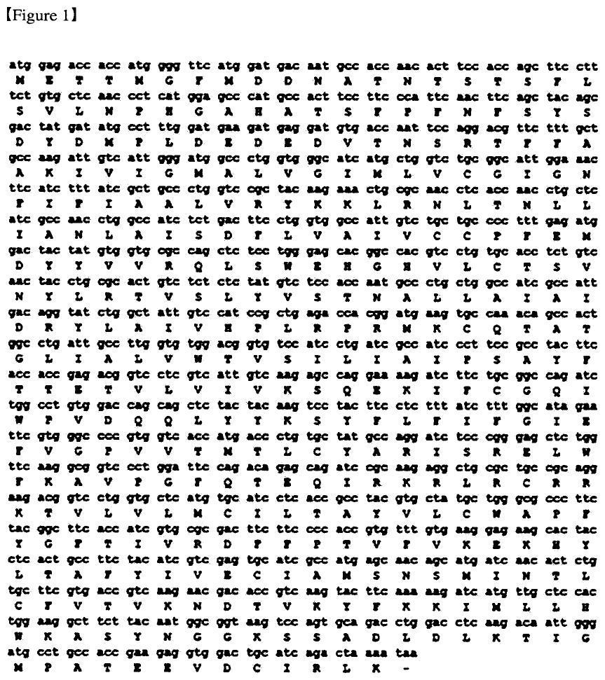

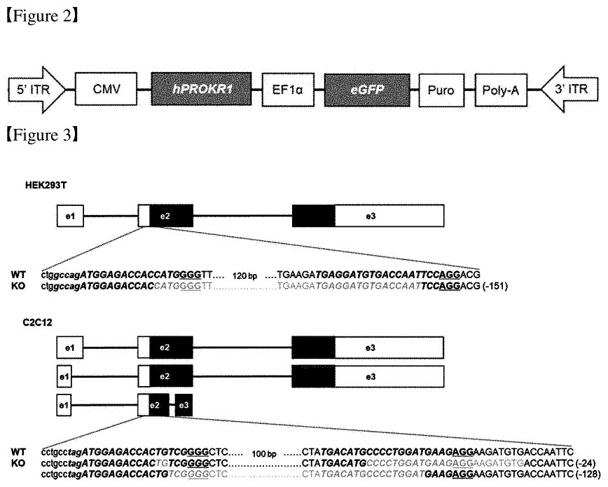

The PROKR1 gene was inserted into the overexpression vector through Not I restriction enzyme treatment and cloning, and it was checked whether the overexpression vector of the PROKR1 gene was produced through nucleotide sequence analysis and gene cloning. cDNA sequence information of the human PROKR1 gene (SEQ ID NO: 1) and its amino acid sequence information (SEQ ID NO: 2) are shown in FIG. 1, and the PROKR1 overexpressing vector is schematically shown in FIG. 2.

### Example 2. Construction of the PROKR1 Gene-Overexpressing Cell Line

The human PROKR1-overexpressing vector was transferred to human embryonic kidney cell line 293T (HEK293T) cells, and stably expressed cells were selected. HEK293T cells were prepared by using Dulbecco's Modified Eagle Medium (DMEM) basal medium supplemented with 10% (v/v) fetal bovine serum (Gibco BRL, USA) and 1% (v/v) antibiotics (Invitrogen, USA) at 37° C., 5% CO2 incubator. After HEK293T cells were proliferated to the area of 70% to 80%, the PROKR1-overexpressing vector and piggyBac transposase expressing vector prepared in Example 1 were co-transferred together using Lipofectamine 3000 (Invitrogen). For the transfer of the PROKR1-overexpressing vector, the proliferated HEK293T cells were washed with a phosphate buffer, and it was then exchanged with an antibiotic-free medium. The overexpressing vector transfer solution was prepared by mixing 7.5 μL Lipofectamine 3000 and 2.5 μg overexpressing vector in 250 μL Opti-MEM (Invitrogen). PiggyBac transposase transfer solution was prepared by mixing 10 μL Lipofectamine 3000 and 2.5 μg piggyBac transposase expression vector in 250 μL Opti-MEM (Invitrogen). The prepared transition solution was left at room temperature for 5 minutes after mixing. 500 μL of the final transfer mixture was added dropwise to HEK293T cells and then cultured for 3 hours at 37° C., 5% CO2 incubator. After completion of the transfer of the PROKR1-overexpressing vector, the cells were washed three times with a phosphate buffer, and then culture medium was added thereto. The day after the PROKR1-overexpressing vector transfer, 10 μg/mL puromycin was added to the culture medium to select only HEK293T cells transferred with the PROKR1-overexpressing vector. As a transfer control, HEK293T cells transfected with an empty vector without the PROKR1 gene were selected with puromycin and were then used. In the prepared cells, overexpression of the PROKR1 gene was confirmed through nucleotide sequence analysis and gene expression analysis.

### Example 3. Construction of the PROKR1 Gene-Deficient Cell Line

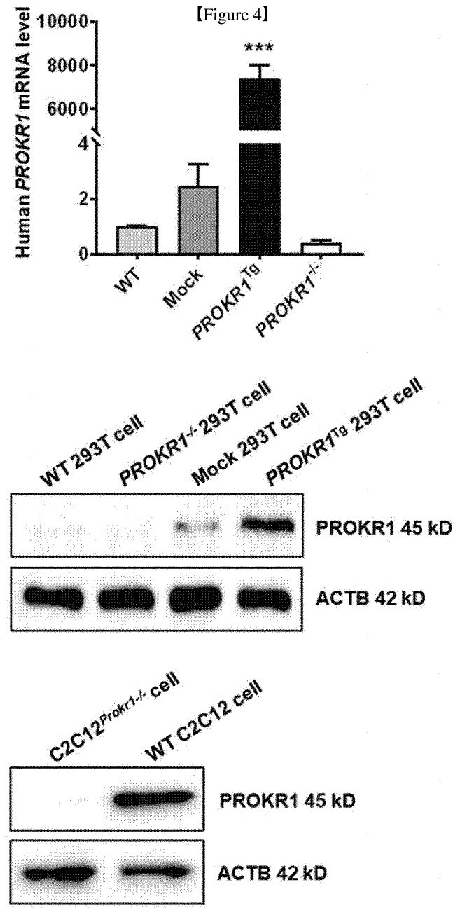

In C2C12 or HEK293T cells, a PROKR1 gene-deficient cell line was constructed using the third-generation gene-editing technique (CRISPR/Cas9). A guide RNA expression vector targeting exon 1 of the PROKR1 gene and a vector expressing Cas9 and green fluorescence protein (GFP) (Sigma, USA) were constructed at the same time, and then each of them was transferred to C2C12 or HEK293T cells using lipofectamine in the same amount of 2.5 μg each. The transferred cells were resuspended in phosphate buffer containing 1% (w/v) bovine serum albumin the next day. The cells were filtered using a 40 μm cell filter (Becton, Dickinson and Company, USA), and then only GFP-positive cells were selected using a fluorescence-activated cell sorter (Becton, Dickinson and Company). In the selected single cells, the deletion of the PROKR1 gene was confirmed through sequencing and gene expression analysis. The nucleotide sequence of the PROKR1 gene-deficient cell is shown in FIG. 3 in which the guide RNA binding site for PROKR1 gene editing is indicated in italics. The expression of the PROKR1 gene in the PROKR1 gene-overexpressing cells prepared in Example 2 and the PROKR1 gene expression of the PROKR1 gene-deficient cells prepared in Example 3 are shown in FIG. 4.

### Example 4. Construction of Transformed Cell-Derived Vesicles

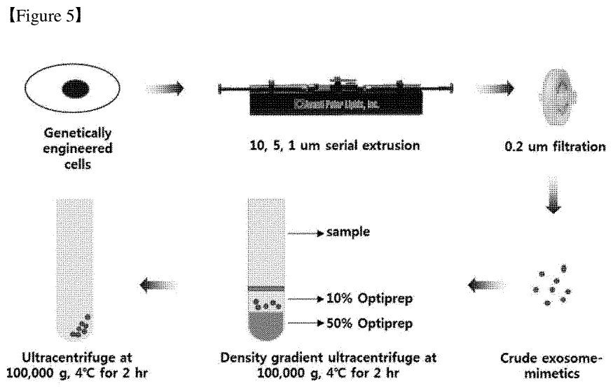

Cell-derived vesicles using cells overexpressing PROKR1 or GFP were prepared using micro-extrusion. Cells proliferated to about 10 million were resuspended in 1 mL of phosphate buffer and then extruded seven times in a micro-extruder provided with the 10 μm, 5 μm, and 1 μm filters. The cell extrudate was finally filtered through a 0.2 μm filter. The cell filtrate was centrifuged by density gradient ultracentrifugation using 10% and 50% (v/v) gradient solutions prepared with OptiPrep medium (Sigma). After density gradient ultracentrifugation, the 10% (v/v) OptiPrep layer was recovered and diluted with phosphate buffer. The result was ultracentrifuged at 4° C. at a speed of 100,000 g for 2 hours to recover the precipitated cell-derived vesicles. The recovered cell-derived vesicles were resuspended in phosphate buffer and stored at −80° C. until the experiment. The manufacturing process of cell-derived vesicles using the micro-extrusion method and density gradient ultracentrifugation as described above is schematically shown in FIG. 5.

### Example 5. Purity and Size Analysis of Cell-Derived Vesicles

5.1 Purity and Size Analysis of GFP-Overexpressing Cell-Derived Vesicles Using Size Exclusive Chromatography (SEC)

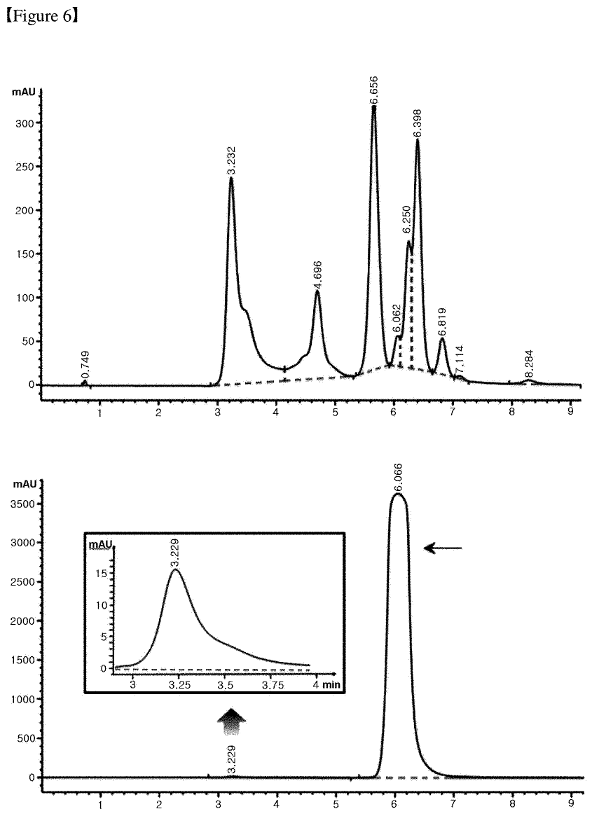

The purity and size of the GFP-overexpressing cell-derived vesicles prepared in the same manner as in Example 4 were analyzed using size exclusion chromatography, and the results are shown in FIG. 6.

As shown in FIG. 6, various peaks were detected in the range of 3 to 7 minutes in the cell-derived vesicle sample before density gradient ultracentrifuge, whereas a single peak was detected at 3.229 minute in the cell-derived vesicle sample after density gradient ultracentrifuge. Therefore, it was confirmed that a high-purity cell-derived vesicle was produced through density gradient ultracentrifuge and the retention time of the prepared cell-derived vesicle was 3.229 minutes.

5.2 Size Analysis of GFP-Overexpressing Cell-Derived Vesicles Using Particle Size Analysis

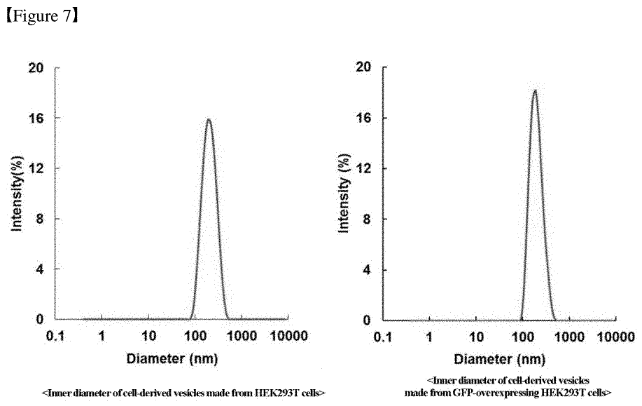

After density gradient ultracentrifuge prepared in the same manner as in Example 4, particle size analysis (Zetasizer, Malvern Panalytical, UK) was performed on the cell-derived vesicles, and the results of the measurement repeated three times are shown in FIG. 7.

As shown in FIG. 7, the inner diameters of cell-derived vesicles prepared from HEK293T and GFP-overexpressing HEK293T cells were measured to be 168±10.2 nm and 173±12.0 nm (mean±standard deviation), respectively. This is a size similar to the inner diameter of exosomes released from cells, and it was confirmed that the cell-derived vesicles produced through micro-extrusion and density gradient ultracentrifuge were similar in size to exosomes naturally released from cells.

### Example 6. Analysis of Marker and Content of GFP-Overexpressing Cell-Derived Vesicles

6.1 Analysis of the Expression of Marker Proteins in GFP-Overexpressing Cell-Derived Vesicles

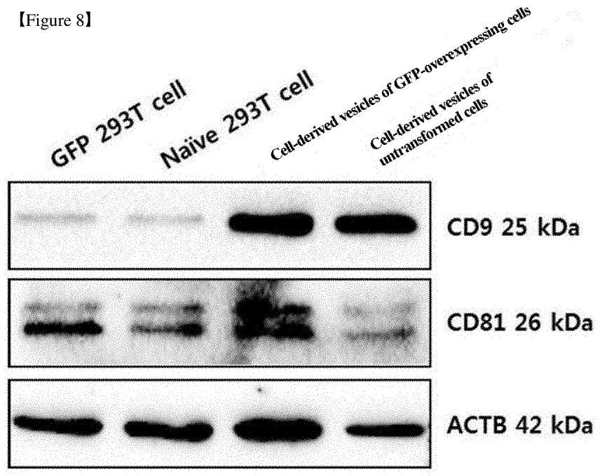

The expression of the marker protein of exosomes was analyzed from the cells used for the preparation of cell-derived vesicles and proteins extracted from cell-derived vesicles. Analysis of the marker protein was performed by Western blot. To confirm whether human anti-CD9 antibody, anti-CD81 antibody, and the same amount of protein solution were analyzed, an anti-beta-actin antibody was used as a control, and the results of Western blot analysis are shown in FIG. 8.

As shown in FIG. 8, the expression of CD9 and CD81 was observed in HEK293T cells and GFP-overexpressing cells. Also, the expression of CD9 and CD81 was confirmed in cell-derived vesicles prepared from each cell, and in the case of CD9, it was confirmed that the protein expression in cell-derived vesicles was higher than that of cells.

6.2 Quantitative Analysis of Protein and RNA Contained in GFP-Overexpressing Cell-Derived Vesicles

For the cell-derived vesicles prepared in the same manner as in Example 4, quantitative analysis was performed on the amount of cell-derived vesicles, the amount of protein and the amount of RNA contained in the cell-derived vesicle. The amount of the prepared cell-derived vesicles was quantified using the Bradford method, and the amount of protein contained in the cell-derived vesicles was quantified by the Bicinchoninic acid method. For the amount of RNA in the cell-derived vesicle, total RNA was recovered using a Trizol solution, and then qualitative and quantitative analysis was performed with a BioAnalyzer, and the analysis results are shown in Table 1.

TABLE 1

| | | Cell-derived vesicles |
|---|---|---|
| | GFP-overexpressing Cell- | derived from |
| | derived vesicles | untransformed cells |
| Bradford (μg) | 101.2 ± 8.9 | 67.5 ± 9.3 |
| BCA (μg) | 104.9 ± 7.6 | 63.3 ± 6.9 |
| RNA (μg) | 13.1 ± 0.9 | 5.5 ± 0.7 |

As shown in Table 1, as a result of a quantitative analysis of cell-derived vesicles, it was confirmed that the amount of cell-derived vesicles prepared from 10 million GFP-overexpressing HEK293T cells was 101.2 μg, and the amount of cell-derived vesicles prepared from 10 million untransformed HEK293T cells was 67.5 As a result of analyzing the amount of protein in the cell-derived vesicles, it was confirmed that the cell-derived vesicle derived from the GFP-overexpressing HEK293T cell contained 104.9 μg of protein, and the cell-derived vesicles derived from untransformed HEK293T cells contained 63.3 μg of protein. In addition, it was confirmed that cell-derived vesicles derived from GFP-overexpressing cells contained 13.1 μg of total RNA, and cell-derived vesicles derived from untransformed cells contained 5.5 μg of total RNA.

6.3 Total RNA Profiling Analysis in Cell-Derived Vesicles

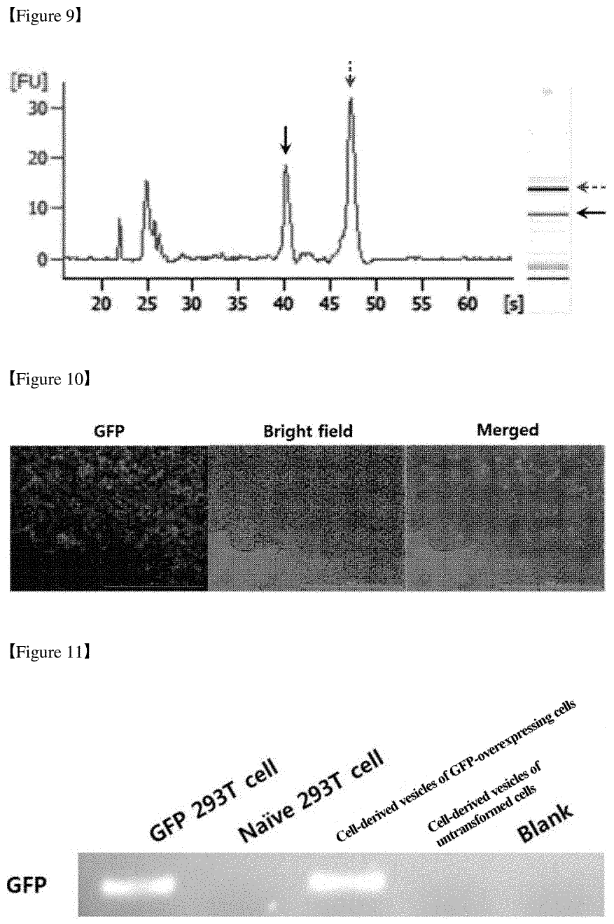

For the cell-derived vesicles derived from the GFP-overexpressing HEK294T cells prepared in the same manner as in Example 4, the results of analyzing the total RNA profile in the cell-derived vesicles are shown in FIG. 9.

As shown in FIG. 9, miRNA and tRNA were observed around 25 seconds, 18S rRNA was observed around 40 seconds, 28S rRNA was observed around 47 seconds, and the absorbance ratio of 18S rRNA and 28S rRNA was observed to be about 1:2. This indicates that RNAs of various sizes are conserved in complete form in the prepared cell-derived vesicles.

### Example 7. Confirmation of Intercellular Transport of Target Substances Using Cell-Derived Vesicles

7.1 Analysis of Target Substances in Cell-Derived Vesicles

GFP expression in cell-derived vesicles derived from GFP-overexpressing HEK293T cells prepared in the same manner as in Example 4 was confirmed. After spreading the cell-derived vesicle sample on a glass slide, it was observed under a fluorescence microscope, and the observation results are shown in FIG. 10.

As shown in FIG. 10, the green fluorescence signal expressed in individual cell-derived vesicles was confirmed. From this, it was confirmed that the target protein, GFP, was contained in a high concentration in the cell-derived vesicle prepared from the GFP-overexpressing cell.

In addition, GFP mRNA was confirmed from the cells used to prepare the cell-derived vesicle and the mRNA recovered from the cell-derived vesicle derived from the corresponding cell, and the results of GFP mRNA expression are shown in FIG. 11.

As shown in FIG. 11, it was confirmed that GFP mRNA was expressed only in GFP-overexpressing HEK293T cells and cell-derived vesicles derived from GFP-overexpressing HEK293T cells.

7.2 Transport of Target Substances by Cell-Derived Vesicles

7.2.1 Confirmation of Transport of GFP Protein by Cell-Derived Vesicles

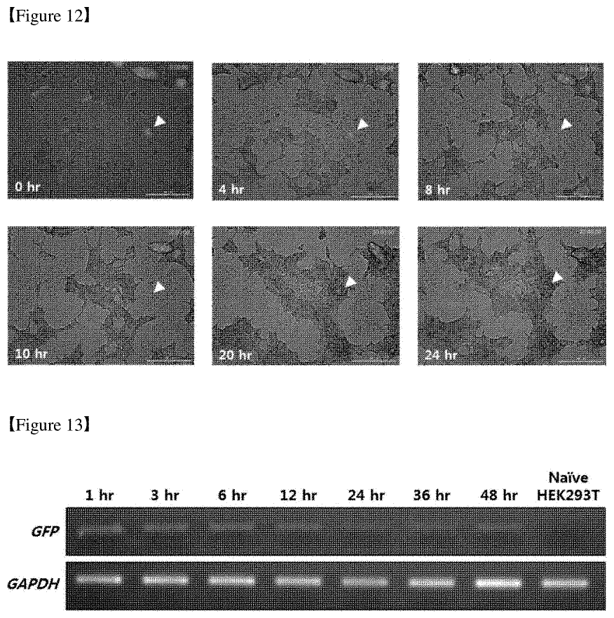

Cell-derived vesicles derived from GFP-overexpressing HEK293T cells prepared in the same manner as in Example 4 were treated to untransformed HEK293T cells, and then the intercellular movement of GFP protein by the cell-derived vesicles was traced, and the tracking results is shown in FIG. 12.

As shown in FIG. 12, it was confirmed that cell-derived vesicles started uptake into HEK293T cells within 30 minutes of treatment into cells and GFP was expressed until 24 hours after treatment. Through these results, it was confirmed that the cell-derived vesicle is smoothly transported to the target cell, and the target protein concentrated in the cell-derived vesicle is also transported to the target cell, and its function is maintained for 24 hours or more.

7.2.2 Confirmation of Transport of GFP mRNA by Cell-Derived Vesicles

After the cell-derived vesicles derived from GFP-overexpressing HEK293T cells prepared in the same manner as in Example 4 were treated to HEK293T cells, and mRNA was recovered from the HEK293T cells for each time period after treatment, it was confirmed whether GFP mRNA remained by RT-PCR. The results are shown in FIG. 13.

As shown in FIG. 13, it was confirmed that GFP mRNA was observed from 1 hour after treatment with cell-derived vesicles derived from GFP-overexpressing HEK293T cells, and GFP mRNA was maintained until 48 hours. Therefore, it was confirmed that cell-derived vesicles prepared from GFP-overexpressing HEK293T cells contained GFP mRNA in addition to GFP protein, and the contained GFP protein and mRNA were absorbed into target cells and maintained for up to 48 hours or longer.

### Example 8. Determination of Treatment Concentration of Cell-Derived Vesicles for Treatment to Cells

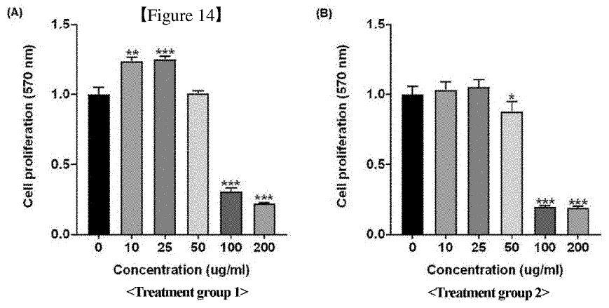

In order to determine the appropriate concentration for treating PROKR1-overexpressing cell-derived vesicles into cells, a dose determination test was performed on C2C12, a rodent-derived myoblast deficient in PROKR1, a target cell. PROKR1-overexpressing cell-derived vesicles were treated at a concentration of 0, 10, 25, 50, 100 and 200 μg/mL for 48 hours. In treatment group 1, the total amount of cell-derived vesicle medium was replaced every 24 hours. In treatment group 2, the same cell-derived vesicle medium was treated for 48 consecutive hours. The results of confirming changes in cell proliferation capacity for C2C12 cells according to the treatment concentration of vesicles derived from PROKR1-overexpressing cells in treatment groups 1 and 2 are shown in FIGS. 14A and 14B, respectively.

As shown in FIG. 14A, in treatment group 1, cell viability of up to a cell-derived vesicle dose group of 50 μg/mL was not significantly different from that of the control group was observed, and cell proliferation inhibition of 60% or more was observed in the dose group of 100 μg/mL or more.

As shown in FIG. 14B, in treatment group 2, significant cell proliferation inhibition was observed from the 50 μg/mL cell-derived vesicle dose group.

Therefore, it was confirmed that the cell-derived vesicle dose of 50 μg/mL was non-toxic to the cells under the condition that the total amount of the cell-derived vesicle medium was replaced at 24 hour intervals.

### Example 9. Size Analysis of PROKR1-Overexpressing Cell-Derived Vesicles

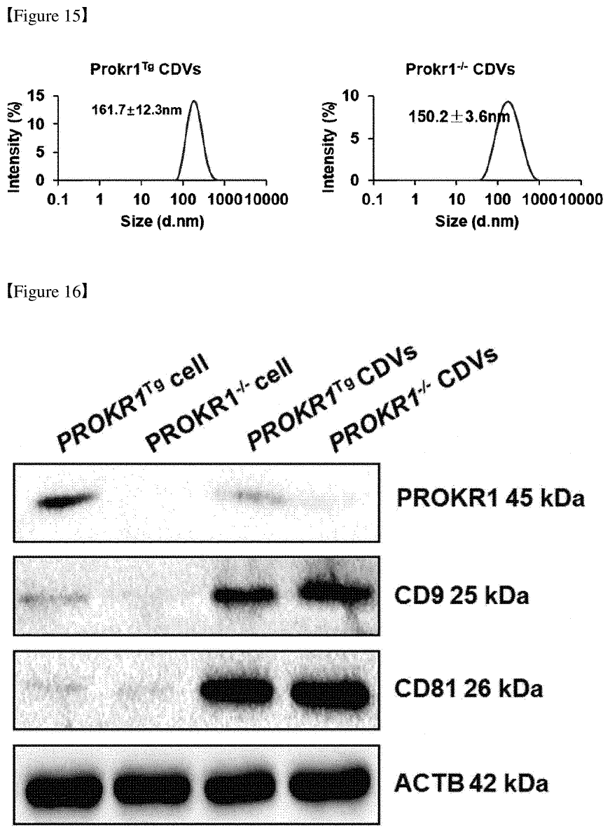

The size distribution of PROKR1-overexpressing cell-derived vesicles or PROKR1-deficient cell-derived vesicles prepared in the same manner as in Example 4 was measured by dynamic light scattering analysis using Zetasizer (Malvern), and the results are shown in FIG. 15.

As shown in FIG. 15, the inner diameters of PROKR1-overexpressing cell-derived vesicles or PROKR1-deficient cell-derived vesicles were measured to be 161.7±12.3 nm and 150.2±3.6 nm (mean±standard deviation), respectively. Accordingly, it was confirmed that the inner diameters of the PROKR1-overexpressing cell-derived vesicles were larger on average than those of PROKR1-deficient cell-derived vesicles

### Example 10. Analysis of Marker and Content of PROKR1-Overexpressing Cell-Derived Vesicles

10.1 Analysis of Expression of Marker Proteins in PROKR1-Overexpressing Cell-Derived Vesicles

Proteins were extracted from the PROKR1-overexpressing cells and PROKR1-deficient cells used for the preparation of cell-derived vesicles and the cell-derived vesicles, and the expression of the marker protein was analyzed. Analysis of the marker protein was performed by Western blot. As primary antibodies, mouse monoclonal anti-β-actin antibody (1:1000, Santa Cruz), rabbit monoclonal anti-CD9 antibody (1:2000), rabbit monoclonal anti-CD81 antibody (1:1000, Abcam) and rabbit monoclonal anti-PKR1 antibody (1:1000, Biorbyt) was used. In addition, for the chemiluminescence measurement of the primary antibody reaction, a goat anti-mouse IgG antibody (1:1000) and a goat anti-rabbit IgG antibody (1:1000, Thermo Fisher Scientific) were used as secondary antibodies. The results of Western blot analysis are shown in FIG. 16.

As shown in FIG. 16, expression of PROKR1 was observed in PROKR1-overexpressing cells and PROKR1-overexpressing cell-derived vesicles, and no expression of PROKR1 was observed in PROKR1-deficient cells and PROKR1-deficient cell-derived vesicles. Further, in the case of CD9 and CD81, 3.7-fold and 5.6-fold higher expression was observed in PROKR1-overexpressing cell-derived vesicles than in PROKR1-overexpressing cells, respectively, and 12-fold and 6.3-fold higher expression was observed in PROKR1-deficient cell-derived vesicles than in PROKR1-deficient cells, respectively.

10.2 Quantitative Analysis of Protein and RNA Contained in PROKR1-Overexpressing Cell-Derived Vesicles

For the PROKR1-overexpressing cell-derived vesicles and PROKR1-deficient cell-derived vesicles prepared in the same manner as in Example 4, quantitative analysis was performed on the amount of cell-derived vesicles, the amount of protein and the amount of RNA contained in the cell-derived vesicle. The amount of the prepared cell-derived vesicles was quantified using the Bradford method, and the amount of protein contained in the cell-derived vesicles was quantified by the Bicinchoninic acid method. For the amount of RNA in the cell-derived vesicle, total RNA was recovered using a Trizol solution, and then qualitative and quantitative analysis was performed with a BioAnalyzer, and the analysis results are shown in Table 2.

TABLE 2

| | Prokr1^{Tg}CDVs | Prokr1-¹-CDVs |
|---|---|---|
| Bradford (μg) | 95.9 ± 5.2 | 85.7 ± 11.7 |
| BCA (μg) | 120.5 ± 20.4 | 87.0 ± 16.7 |
| RNA (μg) | 13.7 ± 2.87 | 10.9 ± 0.83 |

Mean ± SD, N = 3

As shown in Table 2, as a result of a quantitative analysis of cell-derived vesicles, it was confirmed that the amount of cell-derived vesicles prepared from 10 million PROKR1-overexpressing cells was 95.9 μg, and the amount of cell-derived vesicles prepared from 10 million PROKR1-deficient cells was 85.7 As a result of analyzing the amount of protein in the cell-derived vesicles, it was confirmed that the PROKR1-overexpressing cell-derived vesicle contained 120.5 μg of protein and the PROKR1-deficient cell-derived vesicle contained 87.0 μg of protein. In addition, it was confirmed that the PROKR1-overexpressing cell-derived vesicles contained 13.7 μg of total RNA, and the PROKR1-deficient cell-derived vesicle contained 10.9 μg of total RNA.

### Example 11. Confirmation of Transport of PROKR1 Protein Using PROKR1 Overexpressing Cell-Derived Vesicles into Cells

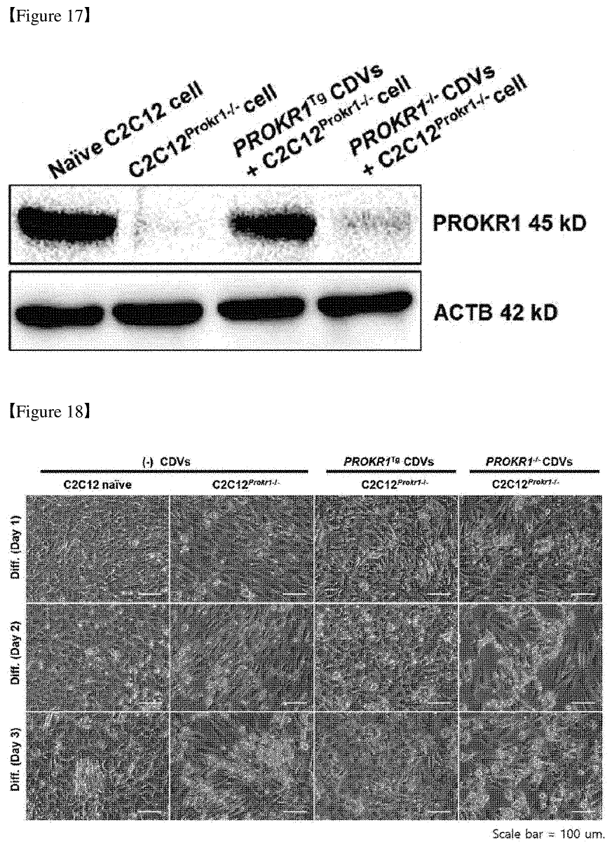

An experiment was performed to determine whether the PROKR1 overexpressing cell-derived vesicles transported the PROKR1 protein into the cell. Untreated C2C12 cell line, PROKR1-deficient C2C12 cell line, PROKR1-deficient C2C12 cell line treated with PROKR1-overexpressing cell-derived vesicle, and PROKR1-deficient C2C12 cell line treated with PROKR1-deficient cell-derived vesicle were set as the experimental group. The expression of PROKR1 was measured in the same manner as in Example 10.1, and the results are shown in FIG. 17.

As shown in FIG. 17, it was confirmed that when the PROKR1-deficient C2C12 cell line was treated with PROKR1-overexpressing cell-derived vesicle, the PROKR1 protein was included in the cell at a high level. From these results, it was confirmed that the PROKR1-overexpressing cell-derived vesicles transported the PROKR1 protein into the cell.

### Example 12. Effects of PROKR1-Overexpressing Cell-Derived Vesicles on the Differentiation of Myoblasts

The effect of the PROKR1-overexpressing cell-derived vesicle on the differentiation of myoblasts was confirmed. Myogenic differentiation was induced with respect to the experimental group set in Example 11, and tissue samples from day 1 to day 3 of induction were observed under a microscope and the observation results are shown in FIG. 18.

As shown in FIG. 18, the formation of myotube was observed in the untreated C2C12 cell line from the third day of induction of myogenic differentiation. Suspension cells were observed in the PROKR1-deficient C2C12 cell line from the first day of induction of myogenic differentiation, and as the induction of myogenic differentiation progressed, the number of suspension cells increased significantly. In comparison, the PROKR1-deficient C2C12 cell line treated with PROKR1-overexpressing cell-derived vesicle significantly reduced the appearance of suspension cells after induction of muscle cell differentiation, and the appearance of early myotube was observed in similar to the myogenic differentiation result of the untreated C2C12 cell line. Meanwhile, the PROKR1-deficient C2C12 cell line treated with PROKR1-deficient cell-derived vesicle showed a similar level of suspension cells to the PROKR1-deficient C2C12 cell line after induction of myogenic differentiation. This confirms that the treatment of PROKR1-overexpressing cell-derived vesicle prevented apoptosis of the PROKR1-deficient C2C12 cell line after induction of myogenic differentiation.

### Example 13. Effects of PROKR1-Overexpressing Cell-Derived Vesicles on Apoptosis after Differentiation of Myoblasts

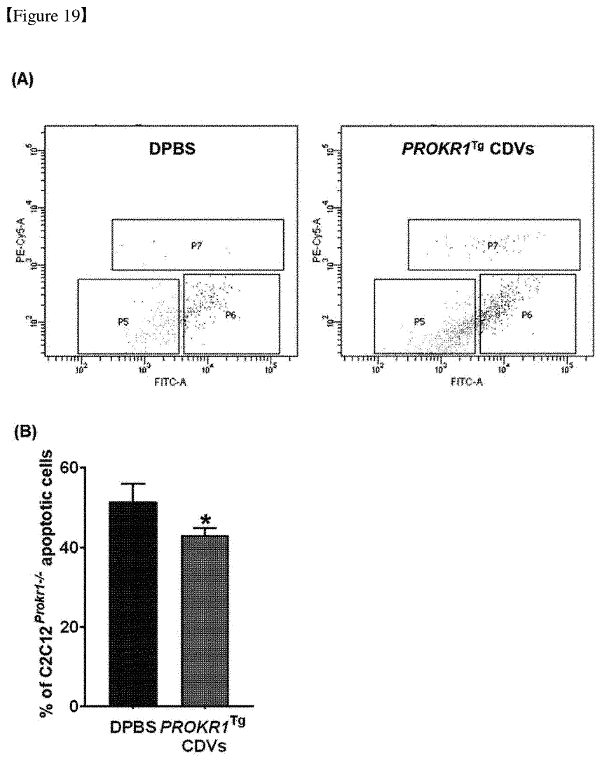

The effect of cell-derived vesicles on apoptosis after differentiation of C2C12 cells, which are myoblasts, was measured through flow cytometry. Pre-differentiated PROKR1-deficient C2C12 cells were treated with PROKR1-overexpressing cell-derived vesicles at a concentration of 25 μg/mL and cultured for 3 days to induce differentiation of C2C12 cells into myotubes. Thereafter, cells were double-stained with Annexin V and propidium iodide, and the stained cells were subjected to flow cytometry analysis (Becton, Dickinson and Company). Then, Annexin V and propidium iodide-positive cells were counted to measure apoptosis of C2C12 cells in which differentiation was initiated. In the apoptosis assay, Annexin V-positive cells were determined as early apoptotic cells, and Annexin V and propidium iodide co-positive cells were determined as late apoptotic cells. As a control group, a group treated with DPBS (Dulbecco phosphate buffered saliine) instead of cell-derived vesicles was used. Flow cytometry was performed through an independent triplicate experiment. The flow cytometry results are shown in FIG. 19A, and the cell death rate relative to the total number of cells is shown in FIG. 19B.

As shown in FIG. 19A, as a result of inducing differentiation of myocytes by treating DPBS alone into pre-differentiated PROKR1-deficient C2C12 cells with DPBS alone, the number of Annexin V and Annexin V/propidium iodide-negative viable cells decreased, whereas the number of viable cells was significantly increased after differentiation into myocytes in the pre-differentiated PROKR1-deficient C2C12 cells treated with PROKR1-overexpressing cell-derived vesicles.

As shown in FIG. 19B, it was confirmed that when pre-differentiated PROKR1-deficient C2C12 cells were treated with the PROKR1-overexpressing cell-derived vesicles, the apoptosis rate was significantly reduced to a level of 8.4%. From these results, it was confirmed that the PROKR1-overexpressing cell-derived vesicles had the effect of increasing the viability of the PROKR1-deficient C2C12 cells.

### Example 14. Effects of PROKR1-Overexpressing Cell-Derived Vesicles on the Differentiation of Myoblasts into Myotube

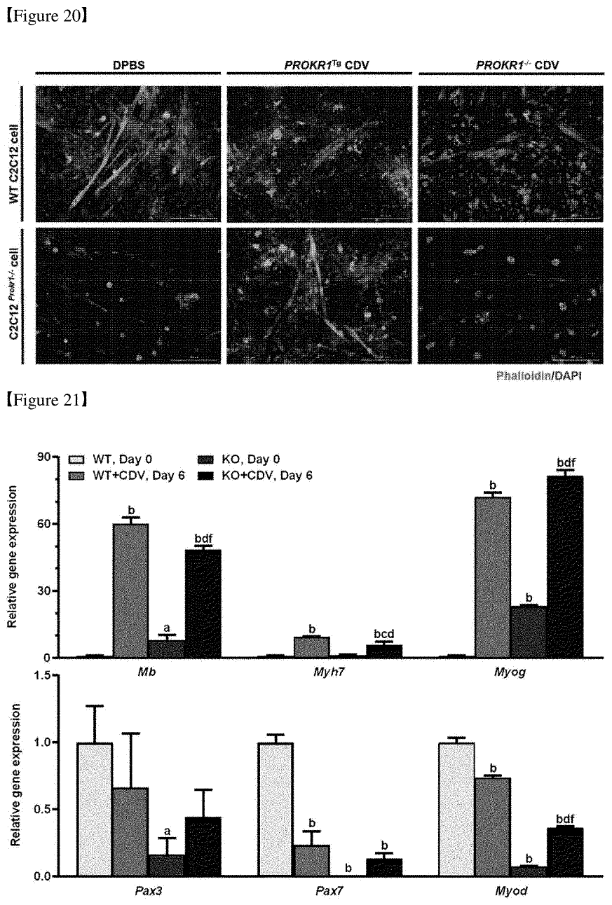

An experiment was conducted to confirm effects of PROKR1-overexpressing cell-derived vesicles on the differentiation of myoblasts into myotube. They were treated with a non-engineered C2C12 cell line or a PROKR1-deficient C2C12 cell line was treated with DPBS, PROKR1-overexpressing cell-derived vesicles, or PROKR1-deficient cell-derived vesicles, followed by phalloidin staining, a myotube-specific actin dye. Specifically, about 10,000 C2C12 cells were cultured and differentiated on a chamber slide, and after 6 days of differentiation into myotube, they were fixed with formalin (Sigma-Aldrich). The fixed cells were perforated with 0.1% (v/v) Triton X-100, blocked with 2% (v/v) BSA, and stained with Alexa Fluor® 488 phalloidin (phalloidin, Thermo Fisher Scientific). Cell nuclei were stained with DAPI. Fluorescence signals of actin and cell nucleus were observed using Cytation 5 (BioTek), and the observation results are shown in FIG. 20.

As shown in FIG. 20, in cells treated with PROKR1-overexpressing cell-derived vesicles, phalloidin-stained myotubes were significantly observed compared to cells treated with DPBS or PROKR1-deficient cell-derived vesicles. From these results, it was confirmed that the PROKR1-overexpressing cell-derived vesicles have the effect of promoting the differentiation of myoblasts into myotube.

### Example 15. Effects of PROKR1-Overexpressing Cell-Derived Vesicle on Myogenic Ability

An experiment was conducted to confirm the effects of PROKR1-overexpressing cell-derived vesicle on myogenic ability. An experimental group in which the untreated C2C12 cell line or the PROKR1-deficient C2C12 cell line were untreated or treated with PROKR1-overexpressing cell-derived vesicles were set, and myogenic differentiation was induced in the same manner as in Example 14. In cells on the 0th and 6th days of induction of myogenic differentiation, the expression of Myh7, Mb, Myog, Pax3, Pax7, and Myod genes, which are myogenesis marker genes, was measured by RT-PCR. Specifically, cell RNA was extracted using RNeasy mini kit (Qiagen), and 5 μg of the extracted RNA was converted into cDNA using Super Script III reverse transcriptase (Invitrogen). For qualitative and quantitative measurement of target mRNA expression from the converted cDNA, real-time PCR systems (Applied Biosystems) were performed using the primers shown in Table 3. The results of RT-PCR are shown in FIG. 21.

TABLE 3

| gene | | | Tm | Amplicon |
|---|---|---|---|---|
| symbol | Forward | Reverse | (° C.) | (bp) |
| Gapdh | AGGTCGGTGTGAACG | TGTAGACCATGTAGTTG | 60 | 123 |
| | GATTTG (SEQ ID NO. 3) | AGGTCA (SEQ ID NO. 4) | | |
| Human- | AGGGCTGCTTTTAACT | CCCCACTTGATTTTGGA | 60 | 206 |
| GAPDH | CTGGT (SEQ ID NO. 5) | GGGA (SEQ ID NO. 6) | | |
| Prokr1 | GCTCTGGTTCGCAGGT | GCAAGGTTGACGACTC | 60 | 119 |
| | TGAA (SEQ ID NO. 7) | CTCT (SEQ ID NO. 8) | | |
| Human- | CAATTCCAGGACGTTC | CTGCGGATGATCTTGTC | 60 | 122 |
| PROKR | TTTGCT(SEQ ID NO. 9) | GGT (SEQ ID NO. 10) | | |
| 1 | | | | |
| GFP | CCGCATCGAGAAGTA | CTGCGGATGATCTTGTC | 60 | 147 |
| | CGAGG (SEQ ID NO. 11) | GGT (SEQ ID NO. 12) | | |
| Myh7 | ACTGTCAACACTAAG | TTGGATGATTTGATCTT | 60 | 114 |
| | AGGGTCA (SEQ ID NO. | CCAGGG (SEQ ID NO. 14) | | |
| | 13) | | | |
| Mb | CTGTTTAAGACTCACC | GGTGCAACCATGCTTCT | 60 | 108 |
| | CTGAGAC (SEQ ID NO. | TCA (SEQ ID NO. 16) | | |
| | 15) | | | |
| Myog | GAGACATCCCCCTATT | GCTCAGTCCGCTCATA | 60 | 106 |
| | TCTACCA (SEQ ID NO. | GCC (SEQ ID NO. 18) | | |
| | 17) | | | |
| Pax3 | TTTCACCTCAGGTAAT | GAACGTCCAAGGCTTA | 60 | 275 |
| | GGGACT (SEQ ID NO. | CTTTGT (SEQ ID NO. 20) | | |
| | 19) | | | |
| Pax7 | TCTCCAAGATTCTGTG | CGGGGTTCTCTCTCTTA | 60 | 132 |
| | CCGAT (SEQ ID NO. 21) | TACTCC (SEQ ID NO. 22) | | |
| Myod | CTACAGTGGCGACTC | GTAGTAGGCGGTGTCG | 60 | 118 |
| | AGA (SEQ ID NO. 23) | TA (SEQ ID NO. 24) | | |

As shown in FIG. 21, on the 6th day of induction of myogenic differentiation in the PROKR1-deficient C2C12 cell line treated with a PROKR1-overexpressing cell-derived vesicle, the mRNA expression levels of late myogenic markers Mb, Myh7 and Myog was observed at a level similar to the corresponding gene expression level of the C2C12 cell line without any manipulation. In addition, on the 6th day of induction of myogenic differentiation in the PROKR1-deficient C2C12 cell line treated with a PROKR1-overexpressing cell-derived vesicle, the expression levels of early myogenic markers Pax3, Pax7 and Myod was observed at a level similar to the corresponding gene expression level of the C2C12 cell line without any manipulation.

### Example 16. Effects of PROKR1-Overexpressing Cell-Derived Vesicles on Glucose Uptake Capacity of Myotube by Insulin Stimulation

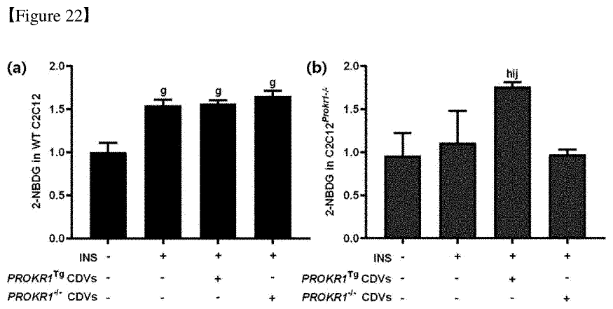

To determine the effect of PROKR1-overexpressing cell-derived vesicles on glucose uptake capacity of myotube by insulin stimulation, 2-[N-(7-nitrobenzene-2-oxa-1,3-diazol-4-yl) amino]-2-deoxy-D glucose (2-NBDG) (Thermo Fisher Scientific) was used to evaluate the glucose uptake capacity of myotube cells by insulin stimulation. The medium of C2C12 cells differentiated into myotube was replaced with a glucose-free DMEM culture medium, and they were treated with 100 nM of insulin (Cell applications, Inc.) for 30 minutes. After insulin treatment, the medium of myotube was replaced with a glucose-free DMEM culture medium, and they were treated with 80 μM of 2-NBDG for 30 minutes. Intracellular 2-NBDG uptake was measured at an absorbance at 535 nm using Cytation 5 (BioTek), and independent triplicate experiments were performed. The measurement result of glucose absorption capacity is shown in FIG. 22.

As shown in FIG. 22A, upon insulin stimulation in a wild-type C2C12 cell line without gene deletion, there was no significant difference in 2-NBDG uptake according to treatment with PROKR1-deficient cell-derived vesicles or PROKR1-overexpressing cell-derived vesicles. Therefore, it was confirmed that wild-type C2C12 exhibited normal glucose uptake capacity without any special side effects, regardless of treatment with PROKR1-deficient cell-derived vesicles or PROKR1-overexpressing cell-derived vesicles.

However, as shown in FIG. 22B, it was confirmed that upon insulin stimulation in a PROKR1-deficient C2C12 cell line treated with PROKR1-overexpressing cell-derived vesicles, 2-NBDG uptake was significantly increased 1.5 times or more compared to the PROKR1-deficient C2C12 cell line and a PROKR1-deficient C2C12 cell line treated with PROKR1-deficient cell-derived vesicles.

These results confirmed that when C2C12 cells with lost differentiation ability were treated with PROKR1 overexpressing cell-derived vesicles, differentiation into myotube having glucose uptake ability by insulin stimulation was promoted. Through the contents confirmed in this example, it was confirmed that the PROKR1-overexpressing cell-derived vesicles were effective in treating diabetes by improving the differentiation rate of muscle cells having insulin sensitivity.

Hereinafter, formulation examples of a pharmaceutical composition for preventing or treating muscle disease, comprising the cell-derived vesicle of the present invention will be described, but it is not intended to limit the present invention, but merely to describe it in detail.

### Formulation Example 1. Preparation of Powder

100 mg of cell-derived vesicle of the present invention

1 g of lactose

The above ingredients were mixed and filled in an airtight cloth to prepare a powder.

### Formulation Example 2. Preparation of Tablets

100 mg of cell-derived vesicle of the present invention

100 mg of corn starch

100 mg of lactose

2 mg of magnesium stearate

After mixing the above ingredients, tablets were prepared by tableting according to a conventional manufacturing method of tablets.

### Formulation Example 3. Preparation of Capsules

100 mg of cell-derived vesicle of the present invention

100 mg of corn starch

100 mg of lactose

2 mg of magnesium stearate

After mixing the above ingredients, the capsules were prepared by filling in gelatin capsules according to a conventional manufacturing method of capsules.

### Formulation Example 4. Preparation of Pills

100 mg of cell-derived vesicle of the present invention

1.5 g of lactose

1 g of glycerin

0.5 g of xylitol

After mixing the above ingredients, it was prepared so as to be 4 g per ring according to a conventional method.

### Formulation Example 5. Preparation of Granules

100 mg of cell-derived vesicle of the present invention

50 mg of soybean extract

200 mg of glucose

600 mg of starch

After mixing the above ingredients, 100 mg of 30% ethanol was added, and they were dried at 60° C. to form granules, and then the granules were filled in a bag.

### Formulation Example 6. Tablet-Type Health Functional Food

15% by weight of Octacosanol powder, 15% by weight of lactose hydrolyzate powder, 15% by weight of soy protein isolated powder, 15% by weight of chitooligosaccharide, 10% by weight of yeast extract powder, 10% by weight of vitamin and mineral mixture, 4.6% by weight of magnesium stearate, 0.2% by weight of titanium dioxide, 0.2% by weight of glycerin fatty acid ester, and 20% by weight of the cell-derived vesicle of the present invention were blended in a conventional method to prepare a tablet-type health functional food.

### Formulation Example 7. Healthy Drink

A healthy drink is prepared by a conventional method by mixing 5% by weight of honey, 3% by weight of fructose, 0.0001% by weight of riboflavin sodium hydrochloride, 0.0001% by weight of pyridoxine hydrochloride, 86.9998% by weight of water and 5% by weight of the cell-derived vesicle of the present invention.

1. A cell-derived vesicle comprising prokineticin receptor 1 (PROKR1) or a gene encoding the same.

2. The cell-derived vesicle of claim 1, wherein the cell-derived vesicle is prepared by introducing a PROKR1 gene to produce a transformed cell; and micro-extruding the transformed cell.

3. The cell-derived vesicle of claim 1, wherein the cell-derived vesicle has the ability to transport a PROKR1 protein or the gene encoding the same into a cell.

4. The cell-derived vesicle of claim 1, wherein the cell-derived vesicle has the ability to promote myogenic differentiation.

5. The cell-derived vesicle of claim 1, wherein the cell-derived vesicle inhibits apoptosis after differentiation into myoblasts.

6. The cell-derived vesicle of claim 1, wherein the cell-derived vesicle promotes glucose uptake of myotube by insulin stimulation.

7. The cell-derived vesicle of claim 1, wherein the cell-derived vesicle contains marker proteins at least three times higher than marker proteins of the derived cell.

8. The cell-derived vesicle of claim 7, wherein the marker protein is CD9 or CD81.

9. A method of improvement in or treating a muscle disease, the method comprising: treating a composition comprising a cell-derived vesicle containing PROKR1 (Prokineticin receptor 1) or a gene encoding the same to an individual in need thereof.

10. The method of claim 9, wherein the cell-derived vesicle has a concentration of 0.0001 μg/mL to 80 μg/mL.

11. (canceled)

12. (canceled)

13. (canceled)

14. (canceled)

15. The method of claim 9, wherein the pharmaceutical composition is for the prevention or treatment of diabetes and muscle disease.

16. The method of claim 9, wherein the cell-derived vesicle is prepared by introducing a PROKR1 gene to produce a transformed cell; and micro-extruding the transformed cell.

17. The method of claim 9, wherein the muscle disease includes at least one selected from the group consisting of mitochondrial myophthy, encephalopathy, lactic acidosis, and stroke-like episodes (MELAS) syndrome, myoclonic epilepsy with ragged red fiber (MERRF) syndrome, Kearns-Sayre syndrome, myopathy, encephalomyopathy, myasthenia, myasthenia gravis, amyotrophic lateral sclerosis, muscular dystrophy, amyotrophia, muscular hypotonia, muscle weakness and muscular rigidity.

18. (canceled)

19. The method of claim 10, wherein the composition is a pharmaceutical composition or a health functional food composition.

20. A method of promoting myogenic differentiation, the method comprising: treating a reagent composition comprising cell-derived vesicle containing PROKR1 (Prokineticin receptor 1) or a gene encoding the same to cells.

21. The method of claim 20, wherein the cells are PROKR1-deficient cells.


SEQUENCE LISTING (24 sequences)

SEQ ID NO: 1 - DNA, 1182 bp, Artificial sequence
  synthetic sequence
  misc_feature: Human PROKR1 cDNA
atggagaccaccatggggttcatggatgacaatgccaccaacacttccaccagcttcctttctgtgctcaaccctcatggagcccatgccacttccttcccattcaacttcagctacagcgactatgatatgcctttggatgaagatgaggatgtgaccaattccaggacgttctttgctgccaagattgtcattgggatggccctggtgggcatcatgctggtctgcggcattggaaacttcatctttatcgctgccctggtccgctacaagaaactgcgcaacctcaccaacctgctcatcgccaacctggccatctctgacttcctggtggccattgtctgctgcccctttgagatggactactatgtggtgcgccagctctcctgggagcacggccacgtcctgtgcacctctgtcaactacctgcgcactgtctctctctatgtctccaccaatgccctgctggccatcgccattgacaggtatctggctattgtccatccgctgagaccacggatgaagtgccaaacagccactggcctgattgccttggtgtggacggtgtccatcctgatcgccatcccttccgcctacttcaccaccgagacggtcctcgtcattgtcaagagccaggaaaagatcttctgcggccagatctggcctgtggaccagcagctctactacaagtcctacttcctctttatctttggcatagaattcgtgggccccgtggtcaccatgaccctgtgctatgccaggatctcccgggagctctggttcaaggcggtccctggattccagacagagcagatccgcaagaggctgcgctgccgcaggaagacggtcctggtgctcatgtgcatcctcaccgcctacgtgctatgctgggcgcccttctacggcttcaccatcgtgcgcgacttcttccccaccgtgtttgtgaaggagaagcactacctcactgccttctacatcgtcgagtgcatcgccatgagcaacagcatgatcaacactctgtgcttcgtgaccgtcaagaacgacaccgtcaagtacttcaaaaagatcatgttgctccactggaaggcttcttacaatggcggtaagtccagtgcagacctggacctcaagacaattgggatgcctgccaccgaagaggtggactgcatcagactaaaataa

SEQ ID NO: 2 - protein, 393 aa, Artificial sequence
  synthetic polypeptide
  MISC_FEATURE: Human PROKR1 protein
METTMGFMDDNATNTSTSFLSVLNPHGAHATSFPFNFSYSDYDMPLDEDEDVTNSRTFFAAKIVIGMALVGIMLVCGIGNFIFIAALVRYKKLRNLTNLLIANLAISDFLVAIVCCPFEMDYYVVRQLSWEHGHVLCTSVNYLRTVSLYVSTNALLAIAIDRYLAIVHPLRPRMKCQTATGLIALVWTVSILIAIPSAYFTTETVLVIVKSQEKIFCGQIWPVDQQLYYKSYFLFIFGIEFVGPVVTMTLCYARISRELWFKAVPGFQTEQIRKRLRCRRKTVLVLMCILTAYVLCWAPFYGFTIVRDFFPTVFVKEKHYLTAFYIVECIAMSNSMINTLCFVTVKNDTVKYFKKIMLLHWKASYNGGKSSADLDLKTIGMPATEEVDCIRLK

SEQ ID NO: 3 - DNA, 21 bp, Artificial sequence
  synthetic sequence
  misc_feature: Gapdh Forward primer
aggtcggtgtgaacggatttg

SEQ ID NO: 4 - DNA, 23 bp, Artificial sequence
  synthetic sequence
  misc_feature: Gapdh Reverse primer
tgtagaccatgtagttgaggtca

SEQ ID NO: 5 - DNA, 21 bp, Artificial sequence
  synthetic sequence
  misc_feature: Human-GAPDH Forward primer
agggctgcttttaactctggt

SEQ ID NO: 6 - DNA, 21 bp, Artificial sequence
  synthetic sequence
  misc_feature: Human-GAPDH Reverse primer
ccccacttgattttggaggga

SEQ ID NO: 7 - DNA, 20 bp, Artificial sequence
  synthetic sequence
  misc_feature: Prokr1 Forward primer
gctctggttcgcaggttgaa

SEQ ID NO: 8 - DNA, 20 bp, Artificial sequence
  synthetic sequence
  misc_feature: Prokr1 Reverse primer
gcaaggttgacgactcctct

SEQ ID NO: 9 - DNA, 22 bp, Artificial sequence
  synthetic sequence
  misc_feature: Human-PROKR1 Forward primer
caattccaggacgttctttgct

SEQ ID NO: 10 - DNA, 20 bp, Artificial sequence
  synthetic sequence
  misc_feature: Human-PROKR1 Reverse primer
ctgcggatgatcttgtcggt

SEQ ID NO: 11 - DNA, 20 bp, Artificial sequence
  synthetic sequence
  misc_feature: GFP Forward primer
ccgcatcgagaagtacgagg

SEQ ID NO: 12 - DNA, 20 bp, Artificial sequence
  synthetic sequence
  misc_feature: GFP Reverse primer
ctgcggatgatcttgtcggt

SEQ ID NO: 13 - DNA, 22 bp, Artificial sequence
  synthetic sequence
  misc_feature: Myh7 Forward primer
actgtcaacactaagagggtca

SEQ ID NO: 14 - DNA, 23 bp, Artificial sequence
  synthetic sequence
  misc_feature: Myh7 Reverse primer
ttggatgatttgatcttccaggg

SEQ ID NO: 15 - DNA, 23 bp, Artificial sequence
  synthetic sequence
  misc_feature: Mb Forward primer
ctgtttaagactcaccctgagac

SEQ ID NO: 16 - DNA, 20 bp, Artificial sequence
  synthetic sequence
  misc_feature: Mb Reverse primer
ggtgcaaccatgcttcttca

SEQ ID NO: 17 - DNA, 23 bp, Artificial sequence
  synthetic sequence
  misc_feature: Myog Forward primer
gagacatccccctatttctacca

SEQ ID NO: 18 - DNA, 19 bp, Artificial sequence
  synthetic sequence
  misc_feature: Myog Reverse primer
gctcagtccgctcatagcc

SEQ ID NO: 19 - DNA, 22 bp, Artificial sequence
  synthetic sequence
  misc_feature: Pax3 Forward primer
tttcacctcaggtaatgggact

SEQ ID NO: 20 - DNA, 22 bp, Artificial sequence
  synthetic sequence
  misc_feature: Pax3 Reverse primer
gaacgtccaaggcttactttgt

SEQ ID NO: 21 - DNA, 21 bp, Artificial sequence
  synthetic sequence
  misc_feature: Pax7 Forward primer
tctccaagattctgtgccgat

SEQ ID NO: 22 - DNA, 23 bp, Artificial sequence
  synthetic sequence
  misc_feature: Pax7 Reverse primer
cggggttctctctcttatactcc

SEQ ID NO: 23 - DNA, 18 bp, Artificial sequence
  synthetic sequence
  misc_feature: Myod Forward primer
ctacagtggcgactcaga

SEQ ID NO: 24 - DNA, 18 bp, Artificial sequence
  synthetic sequence
  misc_feature: Myod Reverse primer
gtagtaggcggtgtcgta